(19) United States
(12) Patent Application Publication
(10) Pub. No.: US 2007/0055227 A1
(43) Pub. Date: Mar. 8, 2007

(54) Probe used for an ocular procedure

(75) Inventors: Steve Khalaj, Laguna Hills, CA (US); Dorin Panescu, San Jose, CA (US); Larry Hood, Laguna Hills, CA (US)

Correspondence Address: IRELL & MANELLA LLP, 840 NEWPORT CENTER DRIVE, SUITE 400, NEWPORT BEACH, CA, 92660, US

(73) Assignee: Refractec, Inc., Irvine, CA (US)

(21) Appl. No.: 11/222,890

(22) Filed: Sep. 8, 2005

(51) Int. Cl. A61B 18/14

(52) U.S. Cl. 606/41

(57) ABSTRACT

A medical device used to denature corneal tissue. The device includes a probe and an applanator that can flatten the cornea. The probe has a plurality of electrodes that extend through apertures of the applanator and into the cornea. The electrodes deliver energy into the cornea to denature corneal tissue. The corneal tissue is denatured in a pattern that can correct for refractive conditions of the eye.

### BACKGROUND OF THE INVENTION

1. Field of the Invention

The present invention relates to a method and apparatus for treating ocular tissue.

2. Prior Art

Techniques for correcting vision have included reshaping the cornea of the eye. For example, myopic conditions can be corrected by cutting a number of small incisions in the corneal membrane. The incisions allow the corneal membrane to relax and increase the radius of the cornea. The incisions are typically created with either a laser or a precision knife. The procedure for creating incisions to correct myopic defects is commonly referred to as radial keratotomy and is well known in the art.

Radial keratotomy techniques generally make incisions that penetrate approximately 95% of the cornea. Penetrating the cornea to such a depth increases the risk of puncturing the Descemets membrane and the endothelium layer, and creating permanent damage to the eye. Additionally, light entering the cornea at the incision sight is refracted by the incision scar and produces a glaring effect in the visual field. The glare effect of the scar produces impaired night vision for the patient.

The techniques of radial keratotomy are only effective in correcting myopia. Radial keratotomy cannot be used to correct an eye condition such as hyperopia. Additionally, keratotomy has limited use in reducing or correcting an astigmatism. The cornea of a patient with hyperopia is relatively flat (large spherical radius). A flat cornea creates a lens system which does not correctly focus the viewed image onto the retina of the eye. Hyperopia can be corrected by reshaping the eye to decrease the spherical radius of the cornea. It has been found that hyperopia can be corrected by heating and denaturing local regions of the cornea. The denatured tissue contracts and changes the shape of the cornea and corrects the optical characteristics of the eye. The procedure of heating the corneal membrane to correct a patient's vision is commonly referred to as thermokeratoplasty.

U.S. Pat. No. 4,461,294 issued to Baron; U.S. Pat. No. 4,976,709 issued to Sand and PCT Publication WO 90/12618, all disclose thermokeratoplasty techniques which utilize a laser to heat the cornea. The energy of the laser generates localized heat within the corneal stroma through photonic absorption. The heated areas of the stroma then shrink to change the shape of the eye.

Although effective in reshaping the eye, the laser based systems of the Baron, Sand and PCT references are relatively expensive to produce, have a non-uniform thermal conduction profile, are not self limiting, are susceptible to providing too much heat to the eye, may induce astigmatism and produce excessive adjacent tissue damage, and require long term stabilization of the eye. Expensive laser systems increase the cost of the procedure and are economically impractical to gain widespread market acceptance and use.

Additionally, laser thermokeratoplasty techniques non-uniformly shrink the stroma without shrinking the Bowmans layer. Shrinking the stroma without a corresponding shrinkage of the Bowmans layer, creates a mechanical strain in the cornea. The mechanical strain may produce an undesirable reshaping of the cornea and probable regression of the visual acuity correction as the corneal lesion heals. Laser techniques may also perforate Bowmans layer and leave a leucoma within the visual field of the eye.

U.S. Pat. Nos. 4,326,529 and 4,381,007 issued to Doss et al, disclose electrodes that are used to heat large areas of the cornea to correct for myopia. The electrode is located within a sleeve that suspends the electrode tip from the surface of the eye. An isotropic saline solution is irrigated through the electrode and aspirated through a channel formed between the outer surface of the electrode and the inner surface of the sleeve. The saline solution provides an electrically conductive medium between the electrode and the corneal membrane. The current from the electrode heats the outer layers of the cornea. Heating the outer eye tissue causes the cornea to shrink into a new radial shape. The saline solution also functions as a coolant which cools the outer epithelium layer.

The saline solution of the Doss device spreads the current of the electrode over a relatively large area of the cornea. Consequently, thermokeratoplasty techniques using the Doss device are limited to reshaped corneas with relatively large and undesirable denatured areas within the visual axis of the eye. The electrode device of the Doss system is also relatively complex and cumbersome to use.

“A Technique for the Selective Heating of Corneal Stroma” Doss et al., Contact & Intraoccular Lens Medical Jrl., Vol. 6, No. 1, pp. 13-17, January-March, 1980, discusses a procedure wherein the circulating saline electrode (CSE) of the Doss patent was used to heat a pig cornea. The electrode provided 30 volts r.m.s. for 4 seconds. The results showed that the stroma was heated to 70° C. and the Bowman's membrane was heated 45° C., a temperature below the 50-55° C. required to shrink the cornea without regression.

“The Need For Prompt Prospective Investigation” McDonnell, Refractive & Corneal Surgery, Vol. 5, January/February, 1989 discusses the merits of corneal reshaping by thermokeratoplasty techniques. The article discusses a procedure wherein a stromal collagen was heated by radio frequency waves to correct for a keratoconus condition. As the article reports, the patient had an initial profound flattening of the eye followed by significant regression within weeks of the procedure.

“Regression of Effect Following Radial Thermokeratoplasty in Humans” Feldman et al., Refractive and Corneal Surgery, Vol. 5, September/October, 1989, discusses another thermokeratoplasty technique for correcting hyperopia. Feldman inserted a probe into four different locations of the cornea. The probe was heated to 600° C. and was inserted into the cornea for 0.3 seconds. Like the procedure discussed in the McDonnell article, the Feldman technique initially reduced hyperopia, but the patients had a significant regression within 9 months of the procedure.

Refractec, Inc. of Irvine Calif., the assignee of the present application, has developed a system to correct hyperopia with a thermokeratoplasty probe that is connected to a console. The probe includes a tip that is inserted into the stroma layer of a cornea. Electrical current provided by the console flows through the eye to denature the collagen tissue within the stroma. The process of inserting the probe tip and applying electrical current can be repeated in a circular pattern about the cornea. The denatured tissue will change the refractive characteristics of the eye. The procedure is taught by Refractec under the service marks CONDUCTIVE KERATOPLASTY and CK.

A CK procedure typically requires a number of single applications with a uni-polar tip. By way of example, a procedure may require 24 separate denatured spots on the cornea. It is desirable to have relatively uniform spacing between denatured spots along the same radian. Sequentially inserting the tip and delivering energy can be a relatively time consuming process. It would be desirable to provide an electrode assembly that can reduce the time required to create the denatured spots in a CK procedure and provide uniform spacing between spots.

It is desirable to insert the electrode at an orientation essentially perpendicular to the surface of the cornea. The cornea has a radius of curvature. The radius makes it difficult to manually insert the electrode perpendicular to the corneal surface, particularly on a repeated basis. It would be desirable to provide a tool and process that would improve the accuracy of electrode insertion into the cornea.

### BRIEF SUMMARY OF THE INVENTION

A probe and an applanator used to denature corneal tissue. The applanator has a plurality of apertures. The probe has a plurality of electrodes that are inserted through the apertures into the cornea.

### BRIEF DESCRIPTION OF THE DRAWINGS

FIG. 1 is a perspective view of a system for denaturing corneal tissue;

FIG. 2 is an enlarged top perspective view of the probe of the system inserted through the applanator;

FIG. 3 is a bottom perspective view of the probe shown in FIG. 2;

FIG. 4 is a view of the probe and the applanator prior to the insertion process;

FIG. 5 is a graph showing a waveform that is provided by a console of the system;

FIG. 6 is an illustration showing an electrode inserted into a cornea;

FIG. 7 is a top view showing a pattern of denatured spots in a cornea;

FIG. 8 is a side cross-sectional view of an alternate embodiment of the probe;

FIG. 9 is a perspective view of an alternate embodiment of a medical device.

### DETAILED DESCRIPTION

Disclosed is a medical device used to denature corneal tissue. The device involves a probe and an applanator that can flatten the cornea. The applanator flattens the cornea so that the electrodes are inserted essentially perpendicular to the outer corneal surface. The probe further has a plurality of electrodes that, when inserted, extend through apertures of the applanator and into the cornea. The electrodes deliver energy into the cornea to denature corneal tissue. The essentially perpendicular insertion of the tips creates a more repeatable procedure. The corneal tissue is denatured in a pattern that can correct for hyperopic conditions of the eye.

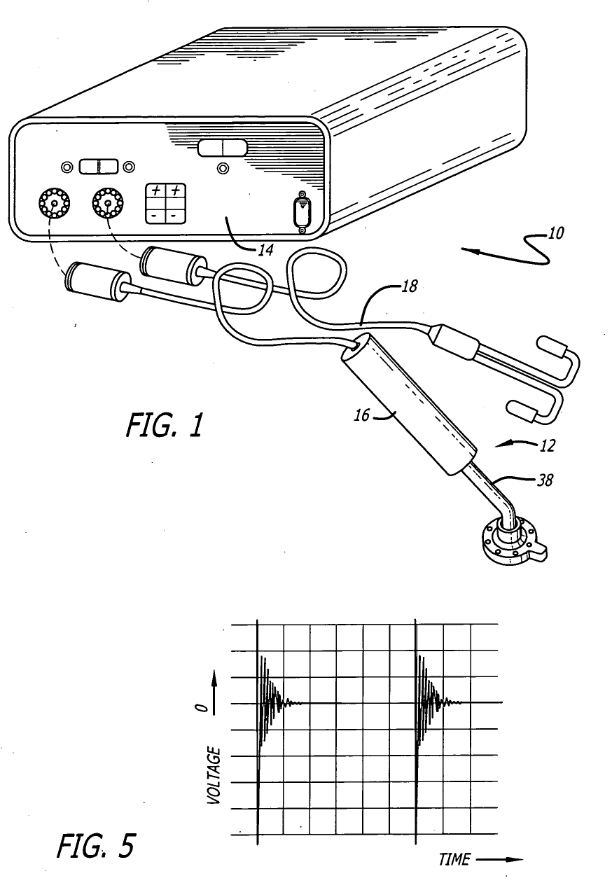

Referring to the drawings more particularly by reference numbers, FIG. 1 shows an embodiment of a system 10 that can be used to denature corneal tissue. The system 10 includes a probe 12 coupled to a console 14. The console 14 contains a power supply that can deliver electrical power to the probe 12. The system 10 may also include a return element 18 that provides a return path for current delivered by the probe 12. By way of example, the return element 18 may be a lid speculum that is used to maintain a patient's eyelids open during a medical procedure.

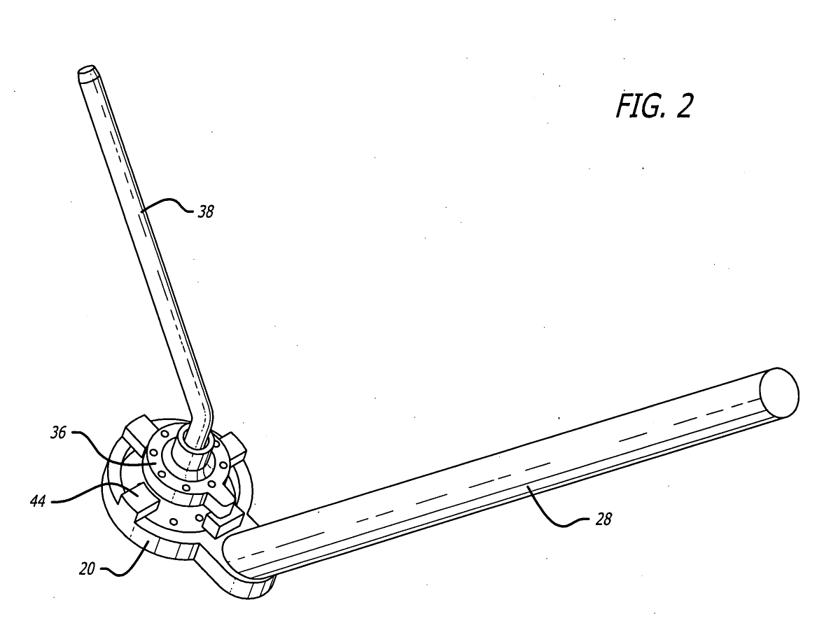

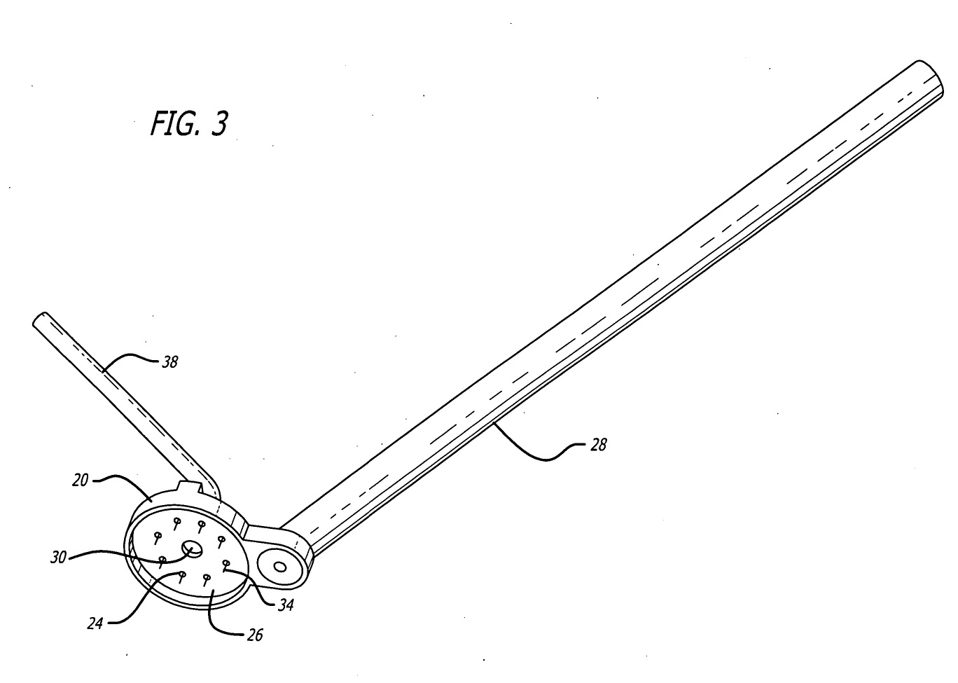

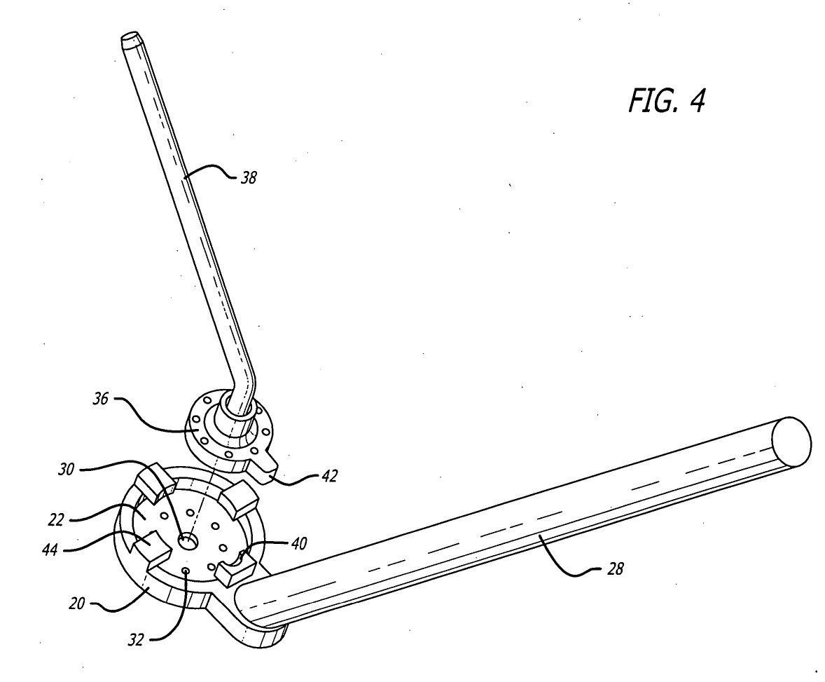

FIGS. 2-4 show an embodiment of the probe 12. The probe 12 works in combination with an applanator 20. The applanator 20 may include an upper cavity 22 and a lower cavity 24. The lower cavity 24 includes a flat bottom surface 26. The flat bottom surface 26 can be used by a surgeon to flatten a cornea. The applanator 20 includes a handle 28 that allows a surgeon to press the flat bottom surface 26 onto a cornea. The applanator 20 may include a center opening 30 that allows the surgeon to see the cornea and center the applanator 20. The applanator 20 may also have a plurality of apertures 32. The apertures 32 may be equally spaced about the bottom surface 26. By way of example, the applanator 20 may have 8 apertures.

The probe 12 may include a plurality of electrodes 34 that extend through the apertures 32 of the applanator 20. In the preferred embodiment, in order to deliver energy to the cornea, the electrodes 34 of probe 12 are inserted through apertures 32 during the procedure. In a different embodiment, the probe 12 and the applanator 20 may form one single assembly. In such an embodiment, the probe 12 and applanator 20 would be provided together and the operator would have to push the electrodes 34 through apertures 32 after the applanator 20 was located in place and pressed to flatten the cornea. In this embodiment, the device may have a spring (not shown) to return the position of the electrodes. The electrodes 34 are connected to the console shown in FIG. 1. Each electrode 34 preferably has a sharp tip that allows for penetration into a cornea. To insure a uniform energy delivery pattern, the tips may all have the same length.

The electrodes 34 may be supported by a guide plate 36. The electrodes 34 may be connected to a connector pin 38 that is plugged into the hand piece 16 and electrically connected to the console 14. The hand piece 16 can be held by a surgeon. The pin 38 may be connected to the guide plate 36 to allow for simultaneous application of energy to the electrodes 34. Alternatively, the probe 12 may include a multi-wire connector that connects electrodes 34 individually through the hand piece 16 to the console 14 to allow for the sequential application of energy to the electrodes 34. The guide plate 36 can be inserted into the upper cavity 22. The applanator 20 may have an alignment feature to align and prevent rotation of the guide plate 36. The alignment feature may be a groove 40 in the applanator 20 that receives a projection 42 of the guide plate 36. The upper cavity 22 may also have stops 44 that inhibit lateral movement of the guide plate 36.

The electrodes 34 are typically constructed from a metal material, such as stainless steel. The applanator 20 and guide plate 36 may be constructed from a dielectric material such as plastic. For example, the dielectric material may be a polyolefin polymer or polycarbonate. Alternatively, the applanator 20 and/or guide plate 36 may be constructed to include a hollow metal filled with a dielectric material. The applanator 20 is preferably constructed to be transparent at least in the center portion to allow the surgeon to visually locate the position of the device relative to the eye and determine when the cornea has been sufficiently flattened by the applanator.

The console 14 may provide a predetermined amount of energy to the electrodes 34, through a controlled application of power for a predetermined time duration. The console 14 may have manual controls that allow the user to select treatment parameters such as the power and time duration. The console 14 can also be constructed to provide an automated operation. The console 14 may have monitors and feedback systems for measuring physiologic tissue parameters such as tissue impedance, tissue temperature and other parameters, and adjust the output parameter of the radio frequency energy source to accomplish the desired results.

In one embodiment, the console 14 provides voltage limiting to prevent arcing. To protect the patient from overvoltage or overpower, the console 14 may have an upper voltage limit and/or upper power limit which terminates power to the probe when the output voltage or power of the unit exceeds a predetermined value.

The console 14 may also contain monitor and alarm circuits which monitor physiologic tissue parameters such as the resistance or impedance of the load and provides adjustments and/or an alarm when the resistance/impedance value exceeds and/or falls below predefined limits. The adjustment feature may change the voltage, current, waveform, crest factor and/or power delivered by the console such that the physiological parameters are maintained within a certain range. The alarm may provide either an audio and/or visual indication to the user that the resistance/impedance value has exceeded the predefined limits. Additionally, the unit may contain a ground fault indicator, and/or a tissue temperature monitor. The front panel of the console 14 typically contains meters and displays that provide an indication of the power, frequency, etc., of the power delivered to the probe.

The console 14 may deliver a radiofrequency (RF) power output in a frequency range of 100 KHz-5 MHz. In the preferred embodiment, power is provided to the probe at a frequency in the range of 350 KHz. The time duration of each application of power to a particular location of tissue can be up to several seconds.

If the system incorporates temperature sensors, the console 14 may control the power such that the target tissue temperature is maintained to no more than approximately 100° C., to avoid necrosis of the tissue. The temperature sensors can be carried by the probe 12, incorporated into the electrodes 34, or attached within proximity to the electrodes 34.

If the system includes an impedance monitor, the power could be adjusted so that the target tissue impedance, assuming a probe 12 with a tip of length 460 um and diameter of 90 um, decreases by approximately 50% from an initial value that is expected to range between 1100 to 1800 ohm/electrode. If two or more electrodes are energized in parallel, the initial impedance values may be less than 1000 ohm. For example, if probe 12 carries 8 electrodes 34 that are energized simultaneously, then the initial overall impedance value is expected to be in the range of ⅛ of the range above, namely 137 to 225 ohm. The console 14 could regulate the power down if, after an initial descent, the impedance begins to increase. Controls can be incorporated to terminate RF delivery if the impedance increases by a significant percentage from the baseline. Alternatively, or additionally, the console 14 could modulate the duration of RF delivery such that delivery is terminated only when the impedance exceeds a preset percentage or amount from a baseline value, unless an upper time limit is exceeded. Other time-modulation techniques, such as monitoring the derivative of the impedance, could be employed. Time-modulation could be based on physiologic parameters other than tissue impedance (e.g tissue water content, chemical composition, etc.)

FIG. 5 shows a typical voltage waveform that is delivered by the probe 12 to the cornea. Each pulse of energy delivered by the probe 12 may be a highly damped sinusoidal waveform, typically having a crest factor (peak voltage/RMS voltage) greater than 5:1. Each highly damped sinusoidal waveform is repeated at a repetitive rate. The repetitive rate may range between 4-12 KHz and is preferably set at 7.5 KHz. Although a damped waveform is shown and described, other waveforms, such as continuous sinusoidal, amplitude, frequency or phase-modulated sinusoidal, etc. can be employed.

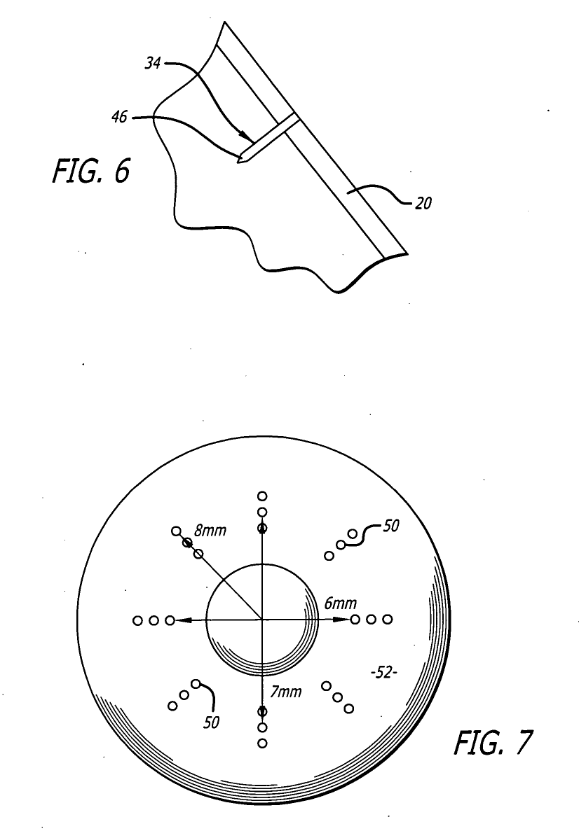

FIG. 6 shows an electrode 34 inserted into a cornea. The pointed tip 46 of the electrode 34 assists in the penetration of the cornea and in the proper distribution of electrical energy to corneal tissue. The applanator 20 flattens the cornea so that the electrode 34 can be inserted in a direction that is essentially perpendicular to the outer cornea surface. The tip 46 is typically inserted until the guide plate 36 becomes fully seated within the upper cavity 22 of the applanator 20.

Each electrode 34 should have a length that insures sufficient penetration into the stroma layer of the cornea. By way of example, the electrodes 34 may each have a length between 300 to 800 microns. The diameter of each electrode 34 should be sufficient to provide the desired amount of energy but be small enough to not leave unsightly incision wounds. In one embodiment, the diameter of each electrode 34 is 90 microns. The electrodes 34 could carry, have embedded in it, or otherwise attached to it, specialized sensors (not shown), such as temperature sensors (e.g. thermocouples, thermistors, etc.), pressure sensors, etc. Although specific lengths and diameters have been disclosed, it is to be understood that the tip may have different lengths and diameters.

In operation, a surgeon pushes the applanator 20 onto a cornea. The applanator 20 flattens the cornea. The applanator may be pressed until a tear film on the cornea extends beyond the electrode aperture. The transparent applanator allows the surgeon to see the tear film move across the bottom applanator surface as the applanator is pressed onto the cornea. The surgeon can then load the guide plate 36 into the upper cavity 22 of the applanator 20. Loading the guide plate 36 inserts the electrodes 34 into the cornea. The surgeon then activates the power unit to deliver energy to the electrodes. The energy flows from the electrodes 34, through the cornea and to the ground element 16. The current generates heat that denatures the collagen tissue of the stroma. The electrodes 34 can deliver the current either sequentially or simultaneously to the cornea.

Because the electrodes 34 are inserted into the stroma, it has been found that a power no greater than 1.2 watts/electrode for a time duration no greater than 1.0 seconds will adequately denature the corneal tissue to provide optical correction of the eye. However, other power and time limits, in the range of several watts and seconds, respectively, can be used to effectively denature the corneal tissue.

FIG. 7 shows a pattern of denatured areas 50 that have been found to correct hyperopic or presbyopic conditions. A circle of 8, 16, or 24 denatured areas 50 are created about the center of the cornea, outside the visual axis portion 52 of the eye. The visual axis has a nominal diameter of approximately 5 millimeters. It has been found that 16 denatured areas provide the most corneal shrinkage and less post-op astigmatism effects from the procedure. The circles of denatured areas typically have a diameter between 6-8 mm, with a preferred diameter of approximately 7 mm. If the first circle does not correct the eye deficiency, the same pattern may be repeated, or another pattern of 8 denatured areas may be created within a circle having a diameter of approximately 6.0-6.5 mm either in line or overlapping.

The applanator 20 may be constructed to accumulate different probes, each probe having a different diameter between electrodes. For example, one probe may have a 6 mm diameter pattern, another probe may have a 7 mm diameter pattern, etc. Alternatively, the probe and applanator may be constructed so that the pattern diameter may be adjustable. The exact diameter of the pattern may vary from patient to patient, it being understood that the denatured spots should preferably be formed in the non-visionary portion 52 of the eye.

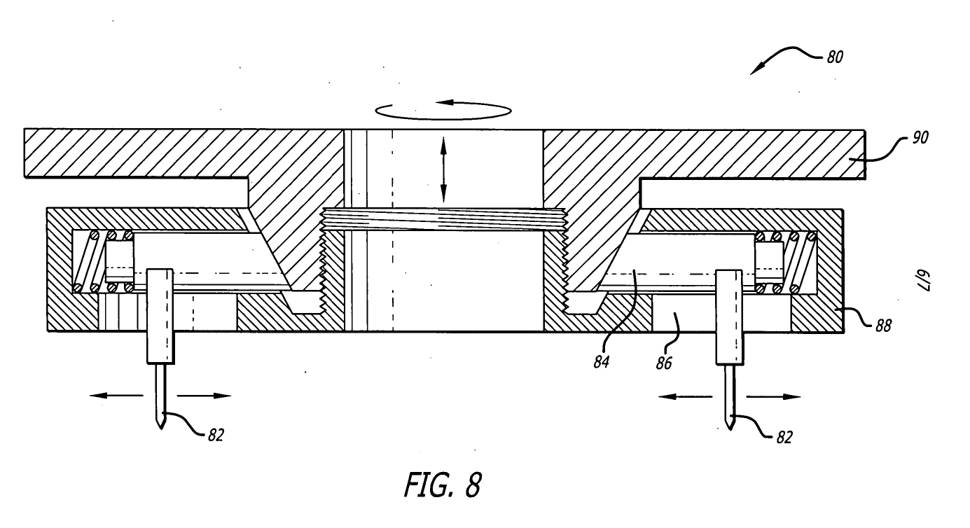

FIG. 8 shows another alternate embodiment of a probe 80. The probe 80 may include a plurality of electrodes 82 attached to a cam plate 84. The electrodes 82 extend through openings 86 of a ring housing 88. The probe 80 may include a knob 90 that is screwed onto the housing 88. Rotating the knob 90 moves the cam plate 86 and varies the position of the electrodes 82. The probe 80 may have springs 92 to provide a biasing force on the cam plate 84. Similarly to the use of probe 12, the probe 80 may be used together with an applanator such as, for example, applanator 20 shown in FIGS. 1, 2, 3, 4, or applanator 102 shown in FIG. 11. Alternatively, probe 80 and applanator 20 could be integrated together into one device.

Although a circular pattern is shown, it is to be understood that the denatured areas may be located in any location and in any pattern. In addition to correcting for hyperopia, the present invention may be used to correct astigmatic conditions. For correcting astigmatic conditions, the denatured areas are typically created at the end of the astigmatic flat axis. The present invention may also be used to correct procedures that have overcorrected for a myopic condition.

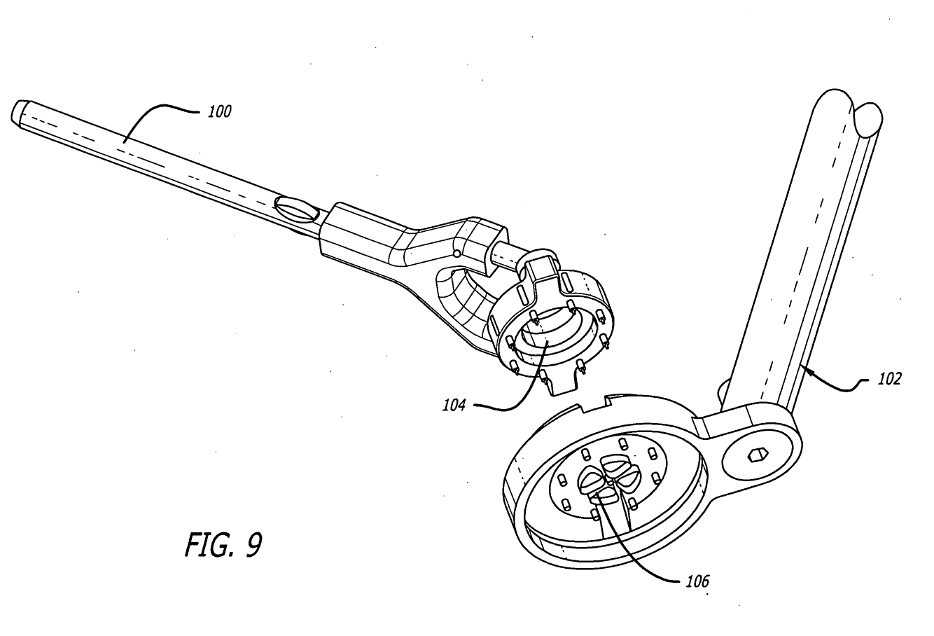

FIG. 9 shows an alternate embodiment of the probe 100 and applanator 102. The probe 100 may have a center opening 104 that allows a surgeon to visually align cross-hairs 106 of the applanator 102 with the cornea.

While certain exemplary embodiments have been described and shown in the accompanying drawings, it is to be understood that such embodiments are merely illustrative of and not restrictive on the broad invention, and that this invention not be limited to the specific constructions and arrangements shown and described, since various other modifications may occur to those ordinarily skilled in the art.

For example, although the delivery of radio frequency energy is described, it is to be understood that other types of non-thermal energy such as direct current (DC), ultrasound and microwave can be transferred into the skin tissue through similar probe concepts.

By way of example, the console can be modified to supply energy in the microwave frequency range or the ultrasonic frequency range. By way of example, the probe may have a helical microwave antenna with a diameter suitable for delivery into the tissue. The delivery of microwave energy could be achieved with or without tissue penetration, depending on the design of the antenna. The system may modulate the microwave energy in response to changes in the characteristic impedance. By way of example, the probe 12 may carry electrodes 34 that contain ultrasonic transducers or piezoelectric vibrators that would transmit ultrasonic energy to the corneal tissue for the purpose of achieving refractive corrections. Additionally, the applanator 20 may be configured and connected to a source of vacuum to create a suction ring that maintains the position of the device relative to the cornea.

What is claimed is:

1. A medical device that is used to denature corneal tissue, comprising:
an applanator that has a plurality of apertures; and,
a plurality of electrodes that extend through said apertures.

2. The device of claim 1, further comprising a guide plate that supports said electrodes.

3. The device of claim 2, wherein said applanator includes an alignment feature that aligns said guide plate with said applanator.

4. The device of claim 2, further comprising a pin that extends from said guide plate and is electrically connected to said electrodes.

5. The device of claim 1, wherein said electrodes each have a tip for insertion into a cornea.

6. The device of claim 3, wherein said alignment feature is a groove that receives a projection of said guide plate.

7. The device of claim 1, wherein there are 8 apertures and 8 corresponding electrodes.

8. The device of claim 1, wherein a portion of at least one electrode is adjustable.

9. The device of claim 1, wherein at least a portion of said applanator is transparent.

10. The device of claim 1, wherein said applanator includes a handle.

11. The device of claim 1, wherein said applanator has an alignment feature for alignment with a cornea.

12. An system that is used to denature corneal tissue, comprising:
an applanator that has a plurality of apertures;
a plurality of electrodes that extend through said apertures; and,
a power unit that delivers energy to said electrodes sufficient to denature tissue of a cornea.

13. The system of claim 12, further comprising a guide plate that supports said electrodes.

14. The system of claim 12, wherein said applanator includes an alignment feature that aligns said guide plate with said applanator.

15. The system of claim 12, further comprising a pin that extends from said guide plate and is electrically connected to said electrodes.

16. The system of claim 12, wherein said electrodes each have a tip for insertion into a cornea.

17. The system of claim 14, wherein said alignment feature is a groove that receives a projection of said guide plate.

18. The system of claim 12, wherein there are 8 apertures and 8 corresponding electrodes.

19. The system of claim 12, wherein a position of at least one electrode pattern is adjustable.

20. The system of claim 12, wherein at least a portion of said applanator is transparent.

21. The system of claim 12, wherein said applanator includes a handle.

22. The system of claim 12, wherein said applanator has an alignment feature for alignment with a cornea.

23. A system that is used to denature corneal tissue, comprising:
applanator means for flattening a cornea;
electrode means for insertion into the cornea and delivery of energy to the cornea to denature corneal tissue; and,
a power unit that provides a sufficient amount of energy to said electrode means to denature the corneal tissue.

24. The system of claim 23, wherein said electrode means includes a guide plate that supports a plurality of electrodes.

25. The system of claim 23, wherein said applanator means includes an alignment feature that aligns said guide plate with said applanator means.

26. The system of claim 23, further comprising a pin that extends from said guide plate and is electrically connected to said electrodes.

27. The system of claim 23, wherein said electrodes each have a tip for insertion into a cornea.

28. The system of claim 25, wherein said alignment feature is a groove that receives a projection of said guide plate.

29. The system of claim 23, wherein said applanator means includes 8 apertures and said electrode means includes 8 corresponding electrodes.

30. The system of claim 23, wherein a position of at least one electrode pattern is adjustable.

31. The system of claim 23, wherein at least a portion of said applanator means is transparent.

32. The system of claim 23, wherein said applanator means includes a handle.

33. The system of claim 23, wherein said applanator means includes an alignment feature for alignment with a cornea.

34. A method for denaturing a cornea, comprising:
flattening a cornea with an applanator;
inserting a plurality of electrodes into the cornea; and,
delivering energy to the cornea through the electrodes to denature corneal tissue.

35. The method of claim 34, wherein the electrodes extend through a plurality of apertures of the applanator.

36. The method of claim 34, wherein the electrodes are inserted by placing a guide plate that supports the electrodes onto the applanator.

37. The method of claim 34, wherein the energy is delivered to the cornea essentially simultaneously.

38. The method of claim 34, wherein the energy is delivered to the cornea essentially sequentially.
